(19) United States
(12) Patent Application Publication
(10) Pub. No.: US 2009/0156881 A1
(43) Pub. Date: Jun. 18, 2009

(54) CONVERGENT WELL IRRADIATING PLAQUE FOR CHOROIDAL MELANOMA

(76) Inventor: John P. Stokes, San Antonio, TX (US)

Correspondence Address: MEYERTONS, HOOD, KIVLIN, KOWERT & GOETZEL, P.C., P.O. BOX 398, AUSTIN, TX, 78767-0398, US

(21) Appl. No.: 12/252,136

(22) Filed: Oct. 15, 2008

Related U.S. Application Data
(60) Provisional application No. 60/980,079, filed on Oct. 15, 2007.

(51) Int. Cl. A61M 36/12; A61F 9/00

(52) U.S. Cl. 600/7

(57) ABSTRACT

Provided in some embodiments is a device suitable for treating an eye that includes a housing and a plurality of fins. The housing includes a base and a rim coupled to the perimeter of the base. The base and the rim at least partially define a cavity in the housing, and the cavity is configured to accept one or more radiation seeds. The plurality of fins at least partially reside within or proximate the cavity of the housing. At least a portion of the fins are configured such that radiation emitted from one or more radiation seeds positioned in the cavity is substantially directed toward a center portion of the eye during use.

### PRIORITY OF THE INVENTION

This application claims priority to U.S. Provisional Patent Application No. 60/980,079 filed on Oct. 15, 2007, which is herein incorporated by reference.

### BACKGROUND OF THE INVENTION

1. Field of the Invention

The present application generally relates to the field of radiation oncology. More specifically, the application discloses a device and method for the treatment of ophthalmic malignancies. In particular, the device and method disclosed herein can be utilized to deliver a dose of radiation to a portion of the eye globe to treat a malignancy of the eye (such as, e.g., choroidal melanoma).

2. Description of the Related Art

Melanoma is a type of cancer that originates within melanocytes, the cells that form pigment or melanin. While melanoma is most commonly found on the skin, it can also occur inside the eye as well as on the surface. The pigmented areas of eye such as the choroid and iris are most commonly affected; however, melanoma sometimes occurs on the conjunctiva as well.

Choroidal melanoma is the most common form of ocular melanoma. The choroid is a highly pigmented layer that lies just behind the retina. With this type of malignancy, the chance of retaining vision in the affected eye is low, but the overall prognosis is often good. The primary concern is the risk of the cancer spreading to another area of the body. The risk is proportional to the size of the tumor, proximity to the optic nerve, visual symptoms, and whether the tumor has documented growth. Those with a tumor that is greater than 2 mm thick or is close to the optic nerve have a higher risk of the melanoma spreading or metastasizing. An individual with none of the above risk factors (e.g., small tumor situated away from the optic nerve, normal vision, and no documented growth over time) may have a very low risk of metastasis.

The appropriate treatment for choroidal melanoma depends largely on the size and location of the melanoma. In general, small tumors that cause no visual symptoms and are not close to the optic nerve may be carefully observed for signs of growth or change. The melanoma is measured and documented with ultrasound, photography, and dilated eye examinations. Small tumors are sometimes treated with laser photocoagulation. Medium and large choroidal melanomas are usually treated either by surgically applying a radioactive plaque to the eye (commonly refered to as episcleral plaque brachytherapy) or by removing the eye completely (enucleation).

Episcleral plaque therapy (brachytherapy) and external-beam, charged-particle radiation therapy offer patients eye-sparing and vision-sparing alternatives to enucleation. Both treatment approaches result in relatively slow regression of uveal melanoma during a period of 6 months to 2 years. Most tumors regress to approximately 50% of their original thickness; only occasionally does a tumor regress to a completely flat scar. Local control is achieved in a large proportion of treated eyes with either technique. The probability of visual preservation and of eye retention with either method is related to tumor size and location.

Episcleral plaque brachytherapy (EBT) is the most frequently used eye-sparing treatment for choroidal melanoma. The goal of EBT is to target radiation to the tumor and spare the eye. If the eye is to be spared, it is important to administer high doses of radiation to the tumor and very little to the rest of the eye. This is typically accomplished by suturing a radioactive ophthalmic plaque to the surface of the eye at the base of the tumor. The ophthalmic plaque consists of radiation seeds fixed to one side of a small disc. One side of the ophthalmic plaque is shielded with a thin layer of gold. Alternatively, the ophthalmic plaque may be shielded by fabricating the plaque of a gold alloy. Gold shielding effectively blocks radiation emitted from the seeds and prevents excessive irradiation of tissues in the head. The tumor is irradiating for a period typically ranging from 3-7 days, after which the ophthalmic plaque is removed.

Iodine-125 (I¹²⁵), gold-198 (¹⁹⁸Au), palladium-103 (¹⁰³Pd), and other ophthalmic plaques can be effective in the treatment of medium-sized melanomas. I¹²⁵ is the most commonly used isotope because of its good tissue penetration, accessibility, adequate shielding of the source, and thus lesser risk to other ocular structures and medical personnel. Methods to ensure proper dose homogeneity to the tumor and plaque placement are critical to successful radiation therapy. Such methods typically include conformal therapy, which seeks to improve dose homogeneity within the tumor while minimizing the dose to uninvolved structures. Radioactive sources are typically distributed uniformly over the surface of an opthalmic plaque and are sometimes offset slightly from the scleral surface in order to reduce the dose to the sclera relative to the apex and prescribed therapeutic margin at the tumor base. Nevertheless, it is not uncommon for scleral dose to exceed the dose to the apex of intermediate to tall tumors by a factor of 4 or more.

Initial results from the Collaborative Ocular Melanoma Study (COMS) have demonstrated comparable 5-year survival rates for patients with medium-sized tumors treated primarily with I¹²⁵ plaque irradiation (5-year survival=82%; 95% CI, 79%-85%) or enucleation (5-year survival=81%; 95% CI, 77%-84%). Among the patients treated with I¹²⁵ brachytherapy, 85% retained their eye for 5 years or more, and 37% had visual acuity better than 20/200 in the irradiated eye 5 years after treatment.

Charged-particle radiation therapy can be performed with a proton beam or helium ions. Some investigators report better tumor control with helium ion irradiation than with I¹²⁵ episcleral plaque treatment in terms of local tumor control and eye retention; however, more anterior segment complications are found.

Other radiation therapy techniques that are occasionally employed but not as extensively studied include external-beam radiation therapy and gamma knife radiation therapy. Preliminary evidence suggests that gamma knife surgery may be a feasible treatment option for medium-sized choroidal melanomas.

Structures and tissues within the eye are highly susceptible to radiation-induced damage. Although every effort is made to minimize the amount of radiation that is delivered to healthy eye tissue adjacent to the melanoma, Iodine-125 plaque radiotherapy is nevertheless associated with significant complications that can lead to loss of visual function or to subsequent enulceation. Complications include cataract formation, neovascularization of the iris, radiation maculopathy, and radiation-induced optic neuropathy. The risk of complications increases with increasing melanoma size. The risk of radiation maculopathy or radiation neuropathy increases with proximity to the macula or optic nerve, respectively [1]. For example, nearly one half of the patients treated with I-125 brachytherapy in the medium-size tumor arm of the COMS lost substantial vision by three years (loss of six or more lines from the baseline).

Modified plaque designs that include partial collimation have been used with success in controlling medium to large choroidal melanomas. However, they have not shown substantially improved results with regard to preservation of vision [4, 5, and 6].

Recently, Ruthenium-106 plaques have shown a lower incidence of side effects, but are used to treat choroidal melanomas of low thickness because of their lower intensity of emitted radiation [7].

Gamma-knife irradiation has been used to treat choroidal melanoma with successful tumor control but poor visual acuity outcome [8].

Other methods of treating choroidal melanoma include:

External-beam, charged-particle radiation therapy: Provides precisely focused radiation with a homogeneous dose distribution pattern and little lateral spread; requires sophisticated equipment available only at selected centers; involves patient cooperation during treatment (voluntarily fixating the eye on a particular point so the tumor is positioned properly in the radiation beam); in eyes with tumors less than 6 mm in thickness and located more than 3 mm distant from the optic disc or fovea, clinically significant visual loss can usually be avoided.

Gamma knife radiation surgery: A newer method of radiation therapy; preliminary experience suggests this treatment may be a feasible option for small-sized to medium-sized melanomas.

Laser photocoagulation: Can be used in very selected cases of small posterior choroidal melanoma; indirect ophthalmoscope laser therapy may be combined with plaque radiation therapy.

Transpupillary thermotherapy: Causes substantial tumor necrosis in choroidal melanomas up to 3.5 mm in thickness; currently used in selected cases with deeply pigmented small choroidal melanomas in the posterior pole with minimal or no contact with the optic nerve; can be used as a primary treatment or as an adjunctive method to plaque radiation therapy

Local eye-wall resection: Good ocular retention rates and visual results have been reported; survival does not appear to be compromised.

Combined therapy, with ablative laser coagulation or transpupillary thermotherapy to supplement plaque treatment: Can be used to minimize recurrence; transpupillary thermotherapy can be used in conjunction with plaque radiation therapy for medium-sized and larger melanomas as an adjuvant treatment to enhance the effects of radiation therapy and to minimize damage to normal ocular tissue; the addition of laser photocoagulation to plaque radiation therapy for juxtapapillary choroidal melanoma has been reported to increase tumor control substantially; ocular side effects do occur but are usually not clinically significant.

Enucleation: Considered primarily if there is a diffuse melanoma or if there is extraocular extension; radiation complications or tumor recurrence may eventually make enucleation necessary.

### SUMMARY OF THE INVENTION

In some embodiments, a device suitable for treating an eye includes a housing and a plurality of fins. The housing includes a base and a rim coupled to the perimeter of the base. The base and the rim at least partially define a cavity in the housing, and the cavity is configured to accept one or more radiation seeds. The plurality of fins at least partially reside within or proximate the cavity of the housing. At least a portion of the fins are configured such that radiation emitted from one or more radiation seeds positioned in the cavity is substantially directed toward a center portion of the eye during use.

In some embodiments, a plurality of fins residing within the cavity of a housing include at least one set of substantially parallel fins. In some embodiments, a plurality of fins residing within the cavity of a housing include a first set of substantially parallel fins in combination with a second set of substantially parallel fins. In an embodiments, a first set of substantially parallel fins are oriented substantially perpendicular to a second set of substantially parallel fins.

In an embodiment, one of the sets of substantially parallel fins are oriented during use such that the longitudinal axis thereof is substantially parallel to the visual axis. In an embodiment, at least a portion of the set of fins that are oriented along the visual axis are angled such that, during use, the planar surface of said fins converge at substantially the center of the eye.

In an embodiment, one of the sets of substantially parallel fins are oriented during use such that the longitudinal axis thereof is substantially perpendicular to the visual axis. In an embodiment, at least a portion of said fins is angled toward the anterior portion of the eye during use.

In one embodiment, a method of treating an eye includes providing radiation to the eye via an eye treatment device. The eye treatment device includes housing and a plurality of fins. The housing includes a base and a rim coupled to the perimeter of the base. The base and the rim at least partially define a cavity in the housing, and the cavity is configured to accept one or more radiation seeds. The plurality of fins at least partially reside within or proximate the cavity of the housing. At least a portion of the fins are configured such that radiation emitted from one or more radiation seeds positioned in the cavity is substantially directed toward a center portion of the eye during use.

In yet another embodiment, a method, includes affixing a treatment device to a surface of an eye. The treatment device includes a plurality of fins disposed between one or more radiation seeds housed within the treatment device and the surface of the eye. At least two of the fins are configured such that radiation emitted from one or more radiation seeds positioned in the cavity is substantially directed toward the center of the eye during use.

In one embodiment, a method of treating an eye includes directing radiation toward and eye using one or more fins.

In one embodiment, system for treating an eye includes a base, a rim, and one or more fins configured to direct radiation toward the eye.

### BRIEF DESCRIPTION OF THE DRAWINGS

Other objects and advantages of the invention will become apparent upon reading the following detailed description and upon reference to the accompanying drawings in which:

FIG. 1A is a perspective view of an ophthalmic plaque in accordance with one embodiment;

FIG. 1B is a perspective view of a housing for use with an ophthalmic plaque in accordance with one embodiment;

FIG. 1C is a cross sectional view of a housing for use with an ophthalmic plaque in accordance with one embodiment;

FIG. 1D is a perspective view of a housing for use with an ophthalmic plaque in accordance with an alternate embodiment;

FIG. 1E is a view along the longitudinal axis of a fin for use with an ophthalmic plaque in accordance with one embodiment;

FIG. 2A is an overview of a set of fins for use with an ophthalmic plaque in accordance with one embodiment;

FIG. 2B is an overview of an alternate embodiment of a set of fins for use with an ophthalmic plaque in accordance with one embodiment;

FIG. 2C is an overview of a further alternate embodiment set of fins for use with an ophthalmic plaque in accordance with one embodiment;

FIG. 3A is a cross sectional view of an ophthalmic plaque for use in accordance with an embodiment;

FIG. 3B is a cross sectional view of an ophthalmic plaque for use in accordance with an alternate embodiment;

FIG. 4 is a perspective view a perspective view of an ophthalmic plaque in accordance with an embodiment;

FIG. 5 is a schematic diagram depicting the positioning of an ophthalmic plaque in accordance with an embodiment;

FIG. 6A is a perspective view of a housing for use with an ophthalmic plaque in accordance with one embodiment;

FIG. 6B is a perspective view of an ophthalmic plaque in accordance with one embodiment;

FIG. 6C shows an overhead view of an ophthalmic plaque for use in accordance with an alternate embodiment;

FIG. 7 is a schematic diagram depicting the positioning of an ophthalmic plaque in accordance with an alternate embodiment;

FIG. 8 is a schematic diagram depicting the positioning of an ophthalmic plaque in accordance with a further alternate embodiment;

FIG. 9A is a schematic diagram of a cross sectional view showing the positioning of an ophthalmic plaque in accordance with an alternate embodiment; and

FIG. 9B is a schematic diagram of a cross sectional view perpendicular to that shown in FIG. 9A, showing the positioning of an ophthalmic plaque in accordance with an alternate embodiment.

FIG. 10 illustrates fins of an ophthalmic plaque in accordance with one embodiment.

While the invention is susceptible to various modifications and alternative forms, specific embodiments thereof are shown by way of example in the drawings and will herein be described in detail. It should be understood, however, that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

The present disclosure generally concerns ophthalmic plaques that are suitable for use in performing an ophthalmic brachytherapy procedure on a subject, as well as methods of using such ophthalmic plaques.

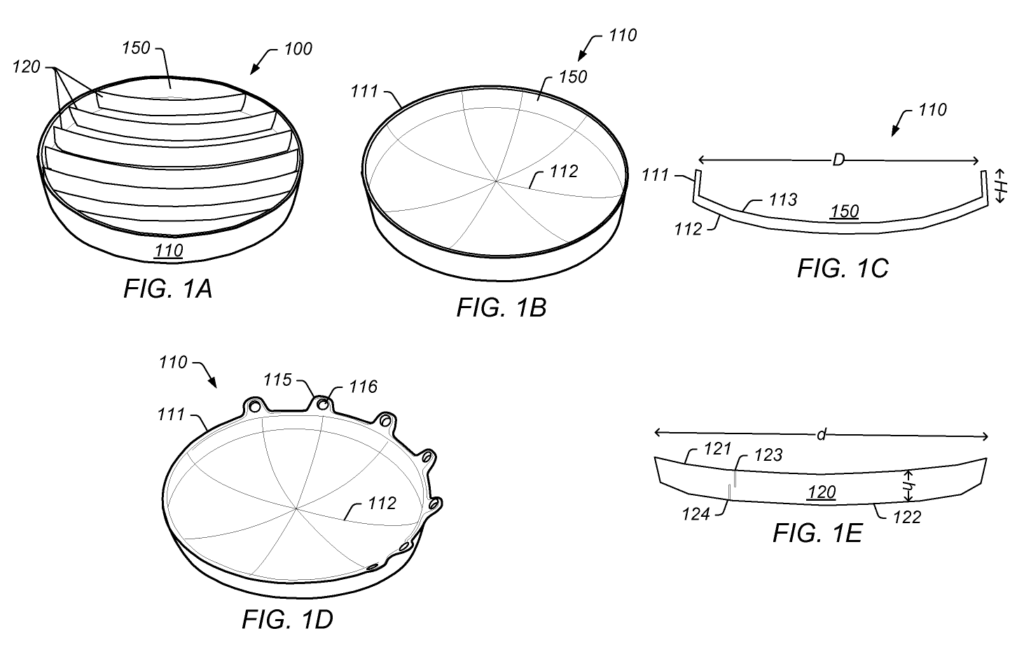

FIG. 1 shows various schematic representations of ophthalmic plaques suitable for use in performing ocular brachytherapy according to the presently described embodiments. Turning to FIG. 1A, ophthalmic plaque 100 may include housing 110 and a plurality of fins 120 positioned within cavity 150 defined within the housing. The plurality of fins 120 may include two or more fins which span cavity 150. The two fins may be oriented substantially parallel to each other. The fins comprising the plurality of fins may have different lengths. The fins used in accordance with the present invention are described in greater detail below.

Housing 110 may be configured such that the plurality of fins may be housed within cavity 150 during use. The length of each fin 120 may vary such that it substantially spans cavity 150 when positioned therein. Housing 110 may include base 112 and rim 111 coupled to the perimeter of the base, thereby defining cavity 150, as depicted in FIG. 1B, which depicts a perspective view of a housing in accordance with certain embodiments of the present invention. Cavity 150 is sized such that the plurality of fins 120 may reside therein. Cavity 150 may further be configured such that one or more radiation seeds (with or without an appropriate seed carrier) may reside therein during use. In one embodiment, the plaque 100 may include a plurality of cavities 150 defined by the housing 110 and/or the fins 120.

The shape of base 112 substantially determines the shape of ophthalmic plaque 100, which may be circular, rectangular, parallelogram, trapezoidal, notched, oval, kidney, or irregularly shaped. The shape of the plaque may be selected to optimize coverage of the base of the tumor to be treated. The diameter of ophthalmic plaque 100 may vary according to the particular needs of the patient who is to undergo ocular brachytherapy. Typically, the diameter of ophthalmic plaque 100 may be at least as large, or preferably larger, than the largest basal diameter of the tumor to be treated. COMS-style plaques may be designed to be about 4 mm larger than the basal diameter of the tumor the being treated. Such a design helps to ensure that an extra 2 mm wide band of treatment beyond the base of the tumor at every point around the circumference of the ophthalmic plaque will be present. The inclusion of such a band may minimize the impact of factors such as improper plaque placement, inadvertent slippage of the plaque, etc. on treatment efficiency. COMS-style plaques may be circular with diameters of 12, 14, 16, 18, and 20 mm.

Turning to FIG. 1C, a profile view of a cross section of a housing according to one embodiment is shown. Housing 110 may be defined by coupling the perimeter of base 112 to rim 111. The height of rim 111 (shown as H in FIG. 1C) in the range of about 0.5 mm to about 5 mm, in the range of about 1 mm to about 4.5 mm, in the range of about 1.5 mm to about 4 mm, or may be about 0.5 mm. Of course, it will be readily apparent to one having ordinary skill in the art that the preceding height ranges are provided merely by way of illustration and are in no way meant to limit the absolute height of a rim that may be employed in the present embodiments. Other rim heights may be employed depending on the requirements of the specific application without departing from the spirit and scope of the present disclosure. Further, the height of rim 112 may not be constant, but rather it may vary at different points along the perimeter of base 112, as will be described in greater detail below.

In an embodiment, base 112 to which rim 111 is coupled may be substantially circular in shape. The diameter of base 112 (shown as D in FIG. 1C) may be selected such that it is at least as large as the base of the tumor being treated. In one embodiment, D is selected such that the base of the ophthalmic plaque covers the entire basal aspect of the tumor, as well as a thin band of surrounding tissue. The inclusion of the thin band in the region to be irradiated helps to ensure that the tumor receives the full radiation dose in the event that the plaque is not precisely placed at the base of the tumor, or in case the plaque moves after being placed. Because the present plaque design minimizes the radiation dose delivered to lateral tissue, a wider band of treatment that extends beyond the base of the tumor may be used, thereby improving the odds that the plaque covers the entire base of the tumor, thus improving treatment efficiency. In general, D will be selected such that a band of up to about 3 mm of healthy tissue surrounding the base of the tumor is covered by the base of the plaque and thus subject to irradiation during the brachytherapy procedure.

In some embodiments, the diameter of ophthalmic plaque 100 may be up to about 10 mm, more preferably between about 4 mm to about 8 mm, and most preferably between about 5 mm to about 6 mm larger than the basal diameter of the tumor the being treated. This will allow for an extra 3 mm or so wide treatment band to ensure that the entire basal area of the melanoma is well treated. Thus, by way of non-limiting example, the diameter of an ophthalmic plaque in accordance with one or more of the presently described embodiments used to treat a tumor having a basal diameter of about 12 mm would be about 18 mm.

In one embodiment, the diameter of ophthalmic plaque 100 may be in the range of about 6 mm to about 30 mm, more preferably in the range of about 12 mm to about 24 mm, and most preferably in the range of about 14 mm to about 22 mm. In certain embodiments, ophthalmic plaque 100 maybe made available in standardized diameters of about 10 mm, about 12 mm, about 14 mm, about 16 mm, about 18 mm, about 20 mm, or about 22 mm. In other embodiments, the ophthalmic plaques may be made to order, having a specified diameter in the range set forth above. Of course, as will be readily apparent to one having ordinary skill in the art that the preceding diameter ranges are provided merely by way of illustration and are in no way meant to limit the dimensions of ophthalmic plaques that may be employed in the present embodiments. On the contrary, the skilled practitioner will recognize that other diameter dimensions may be employed depending on the requirements of the specific application, without departing from the spirit and scope of the present disclosure.

Returning to FIG. 1C, in some embodiments, base 112 may be made to have a substantially concave shape. Base 112 may be coupled to rim 111 such that concave surface 113 faces cavity 150 defined thereby. The thickness of base 112 and or rim 111 may be about 0.5 mm, in the range of about 0.3 to about 0.7 mm, or in the range of 0.4 to about 0.6 mm. As illustrated in the embodiment depicted in FIG. 1C, rim 111 and base 112 need not be of equal thickness. Rim 111 may be thinner or thicker than base 112. Alternatively, the thickness of rim 111 and base 112 may be substantially similar.

FIG. 1D depicts an alternate embodiment of housing 110, in which a plurality a suture eyelets 115 are coupled to the peripheral edge of rim 111. The eyelets are provided to facilitate the placement and suturing of the plaque to the conjunctiva at the commencement of radiotherapy on the tumor. Each suture eyelet may include hole 116 sized to accept a standard suture used to the placement of ophthalmic plaques.

Housing 110 may be constructed using a material that substantially blocks radiation emitted from a radiation source that is typically used in brachytherapy procedures (typically in the range of about 40 cGy/hr to about 110 cGy/hr), such radiation sources may include ¹²⁵I, ⁶⁰Co, ²²²Rn, ¹⁰⁶Ru, ¹⁹²Ir and ¹⁰³Pd. In the case of ¹²⁵I, a low-energy isotope that is most commonly used in ophthalmic brachytherapy procedures, housing 110 may be made using a shielding material at least partially constructed using one or more of the shielding metals gold, lead, brass, or alloys thereof, or any other high Z-materials capable of blocking up to about 0.03 MeV irradiative energy. In the case of alloys, the shielding material comprising the housing will contain greater than about 10 wt. %, more preferably greater than about 30 wt. %, and most preferably greater than about 50 wt. % of the shielding metal. In one embodiment, the entire body of the housing may be constructed of such material. In one embodiment, a layer or a sheet of shielding material may cover one or more surfaces of the housing. The thickness of such a layer may vary depending on the composition of the shielding materials, the amount of isotope used, or other parameters such as will be readily apparent to the skilled practitioner. By way of non-limiting example however, the thickness of the shielding material may not exceed the maximum thickness of the base or the rim of the housing (i.e., up to about 0.7 mm thick). The shielding layer may substantially cover the entire outer surface of the housing. Alternatively, the shielding material may substantially cover the inner surface of the housing, lining cavity 150. In one embodiment, the shielding material may substantially cover the entire housing surface.

Housing 110 may be fabricated in accordance with well-established procedures that are themselves well known to the skilled practitioner. Typically, ophthalmic plaques are fabricated in specialized laboratories (such as, e.g., dental studios).

As described above with respect to FIG. 1A, ophthalmic plaque 100 may include a plurality of fins 120 positioned within cavity 150 defined within the housing. In an embodiment, each of the fins defining the plurality of fins 120 may span cavity 150. The two fins may be oriented substantially parallel to each other. In one embodiment, a majority of the fins may be oriented substantially parallel to each other. The fins comprising the plurality of fins may have different lengths, the length of each fin being optimized the span cavity 150. In one embodiment, each of the fins comprising a plurality of fins 120 may be oriented substantially parallel to the other fins, as depicted in FIG. 1A.

Turing now to FIG. 1E, an embodiment of a fin 120 is shown. Fin 120 may include a thin metal sheet having dimensions d and h. While it will be readily appreciated by the skilled artisan that the value of d may vary depending on how fin 120 is positioned to span cavity 150, in general, the assumption that d≦D holds true. In contrast, the value of h is not under such restriction. In one embodiment, h≦H. In another embodiment, h≧H.

The thickness of each fin 120 may be in the range of about 0.1 to about 0.4 mm, in the range of about 0.2 to about 0.3 mm, or about 0.25 mm. In an embodiment, fin 120 may include at least one curved edge 121. The curvature of edge 121 maybe configured such that edge 121 is at least partially complementary to the curvature of the surface of an eye. In an embodiment, fin 120 maybe positioned in cavity 150 such that edge 121 faces away from base 112. Optionally, fin 120 may further include curved edge 122. The curvature of edge 122 may be less than, greater than, or substantially similar to that of edge 121.

In an embodiment, fin 120 may further include at least one slit 123 and/or at least one slit 124 extending from edges 121 and 122, respectively. Slits 123 and 124 may be sized such that two or more fins 120 are couplable to each other.

In an embodiment, a fin may be made using a radiation shielding material, such as that which is set forth above and incorporated herein. In an embodiment, fin 120 may be made using a shielding material comprising one or more of the shielding metals gold, lead, brass, or alloys thereof, or any other high Z-materials capable of blocking up to about 0.03 MeV irradiative energy. In the case of alloys, the shielding material comprising fin 120 may contain greater than about 10 wt. %, more preferably greater than about 30 wt. %, and most preferably greater than about 50 wt. % of the shielding metal. In one embodiment, the entire fin may be constructed of such material. Alternatively, the fin may be made of a different material (e.g., tin, stainless steel, plastic, etc.) and covered with a layer or a sheet of shielding material. The thickness of such a layer may vary depending on the composition of the shielding materials, the amount of isotope used, or other parameters such as will be readily apparent to the skilled practitioner. By way of non-limiting example however, the thickness of the shielding material will generally not exceed the maximum thickness of a fin (i.e., up to about 0.4 mm thick). The shielding layer may substantially cover the entire outer surface of the fin. Alternatively, the shielding material may substantially cover one or both surfaces of the fin. In one embodiment, the shielding material may substantially cover the entire surface of fin 120.

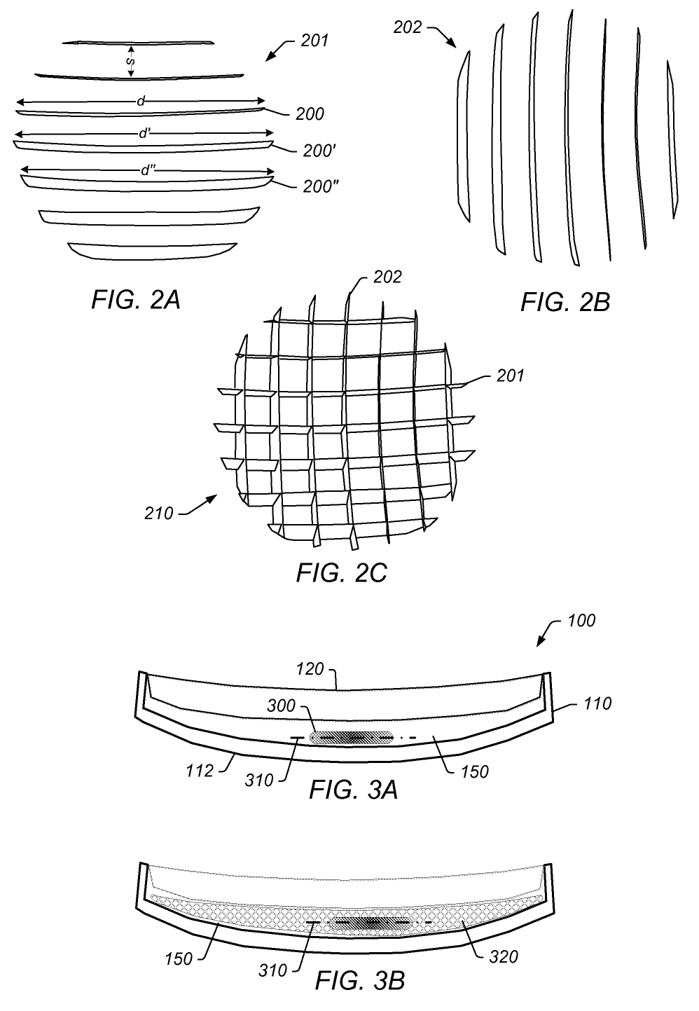

Turning now to FIG. 2, a plurality of fins 200 configured to be positioned in and to span cavity 150 of an ophthalmic plaque as described above is shown. In an embodiment, a plurality of fins 200 may include a first set 201 of fins. The fins comprising a first set 201 may be arranged to be substantially parallel to at least a portion of the fins 200 comprising first set 201, as depicted in FIG. 2A, which shows that fin 200′ is substantially parallel to adjacent fins 200 and 200″. In an embodiment, a first set 201 may be oriented along a specific axis during use. For example, a first set 201 of fins may be positioned in cavity 150 of housing 110 such that the fins comprising first set 201 are substantially parallel to the visual axis during use. For the purpose of the present disclosure, the term “visual axis” generally refers to a straight line extending from the retina to a viewable object that passes through about the center of the pupil and about the center of the fovea.

Each fin 200 has length d, which depends on the position of the fin in cavity 150 of housing 110 when positioned therein. By way of non-limiting example, in the embodiments depicted in FIG. 2A, the length d′ of fin 200′, which passes through the approximate center of cavity 150, is larger than the length d of fin 200, which does not pass through the center of cavity 150.

Still with regard to FIG. 2A, in an embodiment the spacing s of at least a portion of the fins comprising first set 201 may be in the range of about 1.25 mm to about 2.75 mm, or from about 1.5 mm to about 2.5 mm, or from about 1.75 to about 2.25 mm. In an embodiment, at least a portion of the fins 200 comprising a first set 201 may be spaced about 1.75 mm apart. Of course, as will readily apparent to the skilled practitioner, the presently disclosed spacing is provided by way of non-limiting example only, and is in no way intended to limit the scope of the invention. On the contrary, other spacing of the fins may be employed in the practice of the present invention, depending on the specifics of the case (e.g., size of tumor, shape of the plaque, etc.), without departing from the spirit and scope thereof.

FIG. 2B shows an alternate embodiment of a plurality of fins 200 from that depicted in FIG. 2A, wherein a plurality of fins 200 may include a second set 202 of fins. The orientation of second set 202 may differ from that of first set 201. For example, in one embodiment, first set 201 may be oriented such that fins 200 are oriented substantially parallel to the visual axis during use, and second set 202 may be oriented such that fins 200 are oriented substantially perpendicular to the visual axis during use. In one embodiment, a majority of the first set 201 of fins may be oriented substantially parallel to the visual axis during use and a majority of the second set 202 of fins may be oriented substantially perpendicular to the visual axis during use.

FIG. 2C shows a further alternate embodiment of a plurality of fins 210. A plurality of fins 210 may include a first set 201 of fins in combination with a second set 202 of fins. The first set 201 may be oriented differently from second set 202. The fins comprising both the first and second sets may be configured such that first set 201 and second set 202 may be coupled to form a mesh of fins. Plurality of fins 210 maybe configured such that the fins are positionable in cavity 150 of a housing 110 suitable for use in ophthalmic brachytherapy.

In an embodiment, first set 201 may be oriented substantially perpendicular to second set 202, such as is depicted in FIG. 2C. In other embodiments, the first set 201 and second set 202 of fins may be oriented at other angles with respect to one another (e.g., about 30 degrees, 45 degrees, 60 degrees, 75 degrees, or 90 degrees to each other).

Turning now to FIG. 3, a cross sectional view of ophthalmic plaque 100 described in FIG. 1A, with fin 120 spanning cavity 150 of housing 110 is shown. Though not shown in this particular depiction, it will readily apparent to the skilled artisan that fin 120 may be part of a first set 201 of fins, a second set 202 of fins, or plurality of fins 210 such as is disclosed above with respect to FIG. 2A-FIG. 2C.

In one embodiment, at least one radiation seed 300 maybe positioned in cavity 150. For the purposes of the present disclosure, the term “radiation seed” generally refers to any radioactive source material that has been adapted for used in a brachytherapy (in particular ophthalmic brachytherapy) procedure. A variety of suitable radiation seeds are familiar to the person having ordinary skill in art. Exemplary though non-limiting radiation seeds suitable for use with the presently described apparatus, including methods for making and using same, are described in the following U.S. patent references, all of which are hereby expressly incorporated by reference in their entirety as though fully set forth herein: U.S. Pat. Nos. 7,201,715; 7,001,326; 6,926,657; 6,881,183; 6,847,838; 6,820,318; 6,796,936; 6,713,765; 6,712,832; 6,712,782; 6,669,622; 6,666,811; 6,659,933; 6,638,207; 6,635,008; 6,626,817; 6,595,908; 6,582,354; 6,575,898; 6,512,942; 6,503,186; 6,500,109; 6,497,647; 6,471,631; 6,458,068; 6,440,058; 6,419,625; 6,347,443; 6,311,084; 6,206,832; 6,163,947; 6,132,359; 6,129,670; 6,099,457; 6,074,337; 6,066,083; 5,997,463; 5,976,067; 5,713,828; 5,342,283; 5,163,896 and 4,994,013. In general, radiation seeds suitable for use with present apparatus will be rice-sized rods or cylinders having dimensions of <10 mm by <2 mm, or more preferably <5 mm by <1 mm and containing an appropriate dose of ¹²⁵I, ⁶⁰Co, ²²²Rn, ¹⁰⁶Ru, ¹⁹²Ir, ¹⁰³Pd, or their combination. What constitutes an appropriate dose of radioactive material to include in a radiation seed, as well as the number of seeds used and their distribution within an ophthalmic brachytherapy plaque will of course depend on certain variables such as, e.g., the isotope chosen, tumor size, location, height and shape, desired isodose profile, and the general health of the patient. General guidance in determining such variables may be found, for example, at least in the publication by Nag, et al., appearing in “THE AMERICAN BRACHYTHERAPY SOCIETY RECOMMENDATIONS FOR BRACHYTHERAPY OF UVEAL MELANOMAS” 2003, Int. J. Radiation Oncology Biol. Phys., Vol. 56, No. 2, pp. 544-555, which is also hereby expressly incorporated by reference in its entirety as though fully set forth herein.

FIG. 3A depicts an embodiment in which radiation seed 300 is positioned between fin 120 and base 112 of housing 110. Radiation seed 300 may be coupled to upper surface 113 of base 112 using, a small amount of an acrylic fixative, or by any other means of fixing radiation seeds to ophthalmic plaques, such as will be readily apparent to a person having ordinary skill in the art. The radiation seed 300 may be positioned with respect to the visual axis and/or one or more fins 120 during use. For example, in the illustrated embodiment, the radiation seed 300 is oriented such that its longitudinal axis 310 is substantially parallel with the length of the fin 120. In one embodiment, the fin 120 may be positioned substantially perpendicular to the visual axis during use, such that the fin 120 and the radiation seed 300 are substantially perpendicular to the visual axis during use. In another embodiment, the fin 120 may be positioned substantially parallel to the visual axis during use, such that the fin 120 and the radiation seed 300 are substantially parallel to the visual axis during use.

FIG. 3B depicts and alternate embodiment to that shown in FIG. 3A, in which the one or more radiation seeds 300 are held in place by seed carrier 320. In an embodiment, seed carrier 320 may be a modified silastic insert typically used in ophthalmic brachytherapy applications. Such an insert may include a plurality of grooves or slots sized to accept the radiation seeds. Seed carrier 320 may be configured to reside in cavity 150. Optionally, seed carrier 320 may be coupled to upper surface 113 of base 112 using, a small amount of an acrylic fixative. Similar to other described embodiments, the radiation seed 300 may be positioned with respect to the visual axis and/or one or more fins 120 during use.

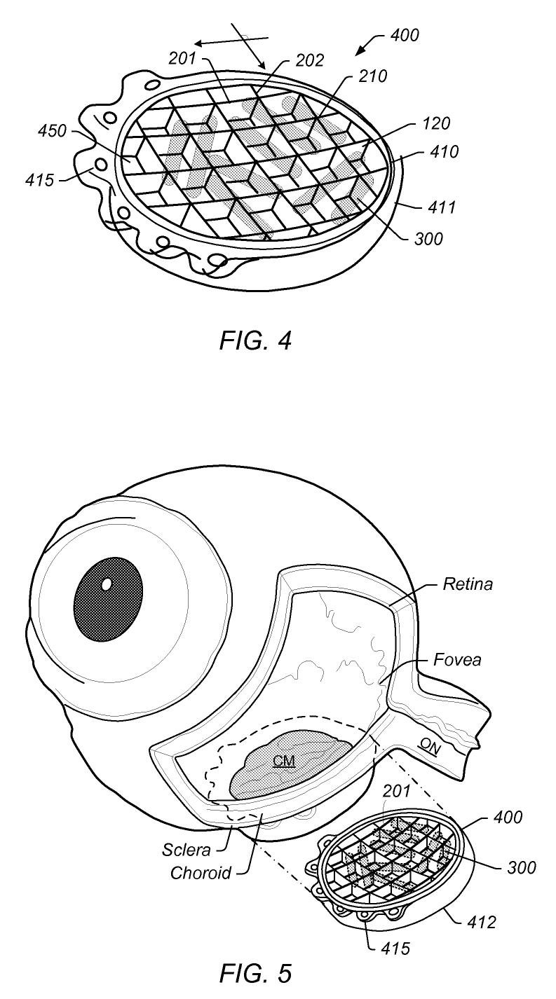

FIG. 4 shows a perspective view of an ophthalmic plaque 400 in accordance with one embodiment. Ophthalmic plaque 400 may include housing 410 having rim 411 and cavity 450 defined thereby. Ophthalmic plaque 400 may further include plurality of fins 210 positioned in cavity 450. Plurality of fins 210 may include first set 201 of substantially parallel fins 120. The fins comprising first set 201 may be oriented substantially parallel to the visual axis during use. Plurality of fins 210 may also include second set 202 of substantially parallel fins 120. The fins comprising second set 202 may be oriented substantially perpendicular to those comprising the first set 201. One or more radiation seeds 300 may be positioned in cavity 450, between plurality of fins 210 and the base (not visible from this angle) of housing 410. Positioning of the fins relative to the radiation seeds, such as is shown in FIG. 4, may substantially reduce the amount of lateral radiation delivered to healthy tissue surrounding the plaque during use. In one embodiment, just one, two, a majority or all of the radiation seeds 300 may be oriented to substantially direct radiation toward a center portion of the eye. Similar to other described embodiments, one or more of the radiation seeds 300 may be positioned with respect to the visual axis and/or one or more fins 120 during use. In an embodiment, one, more than one, a majority of, or all of the radiation seeds 300 may be oriented substantially parallel to the fins 120 of the first set 201 and/or the second set 202 of fins. For example, all of the radiation seeds 300 may be oriented parallel to the fins 120 of the second set 202 of fins, such that during use, the second set of fins 202 and the radiation seeds 300 are oriented substantially perpendicular to the visual axis of the eye.

FIG. 5 shows a cutaway view of an eye showing the position of a choroidal melanoma (CM) relative to the optic nerve (ON), the fovea, and other ocular structures. In an embodiment, ophthalmic plaque 400 may be placed on the surface of the eye directly below the tumor such that the entire base of the tumor is spanned by the plaque (shown as dotted line in FIG. 5).

Plaque 400 may be sutured to the sclera by way of one or more suture eyelets 415. During use, plaque 400 maybe oriented such that the opening of housing 410 and the edges of the fins residing therein face the surface of the eye, whereas base 412 faces away from the surface of the eye, thereby shielding surrounding tissues of the head from radiation emitted by radiation seeds 300. In an embodiment, at least a portion of the fins comprising first set 201 (shown as bolder lines in FIG. 5) may be oriented substantially parallel to the visual axis (defined by a line extending from the fovea through the center of the pupil.

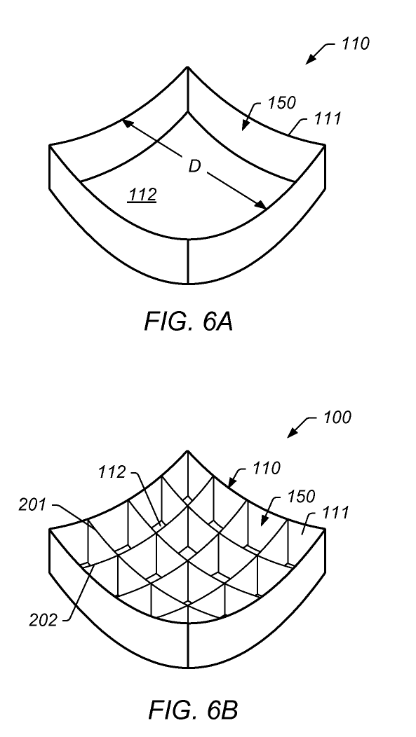

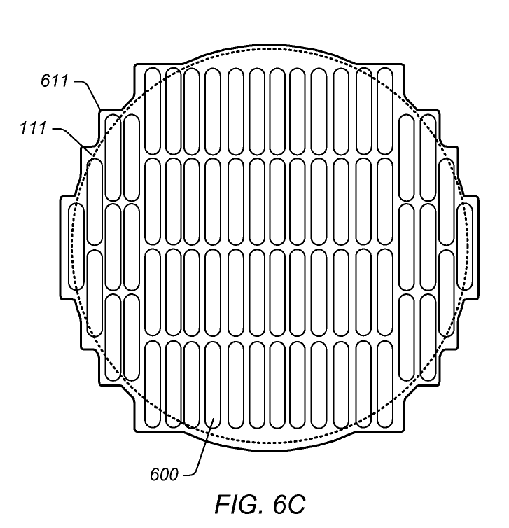

FIGS. 6A-6B illustrate an alternate embodiment of the plaque 100 that includes a substantially rectangular shape. In the illustrated embodiment, the rim 111 and the base 112 to which rim 111 is coupled are substantially rectangular in shape. As depicted in FIG. 6B the plaque 100 may include a plurality of fins 120 disposed in and spanning the cavity 150 of the base 112. Similar to other embodiments described herein, the illustrated rectangular plaque 100 includes a first set 201 and second set 202 of fins. The first set 201 of fins are oriented substantially perpendicular to the second set 202 of fins. In one embodiment, due to the generally rectangular shape, the fins in each set may be of substantially similar length to other fins in the same set. For example, all of the fins in the first set 201 of fins may be of substantially similar length, and all of the fins in the second set 202 of fins may be of substantially similar length. Further, in an embodiment in which the shape of the plaque 100 is substantially a square, the length of each of the fins in the first set 201 and the second set 202 may be of substantially similar length.

The diameter of base 112 (shown as D in FIG. 6A) may include the distance between opposing sides of the rim 111. The diameter 112 may be selected such that it is at least as large as the base of the tumor being treated. In one embodiment, D will be selected such that the base of the ophthalmic plaque covers the entire basal aspect of the tumor, as well as a thin band of surrounding tissue. As depicted, the areas near the comers of the rectangular plaque 100 may extend beyond a radius of length D that extends from the center of the rectangular plaque 100, thereby increasing the total coverage when compared to a circular shaped plaque 100 having the same diameter. Accordingly, the rectangular plaque 100 may prove beneficial in treating tumors of irregular shapes that may be covered by the area of a rectangular shaped plaque 100, and that may not otherwise be covered by a circular plaque 100 of the same diameter D. Similar to the previously discussed embodiment, the inclusion of the thin band in the region to be irradiated helps to ensure that the tumor receives the full radiation dose in the event that the plaque is not precisely placed at the base of the tumor, or in case the plaque moves after being placed.

Embodiments of a rectangular shaped plaque 100 may include any of the features similar to those described herein with respect to other embodiments. For example, the rectangular shaped plaque 100 may include a concave shape, eyelets, material/coatings to block radiation, or the like. Further, certain embodiments may include other shapes. For example, in one embodiment, the base 112 may include a parallelogram, trapezoidal or diamond like shape, wherein the members forming the rim 111 intersect one another at varying angles. In other words, wherein the members forming the rim, and the comers of the base 112 intersect at an acute and/or obtuse angles.

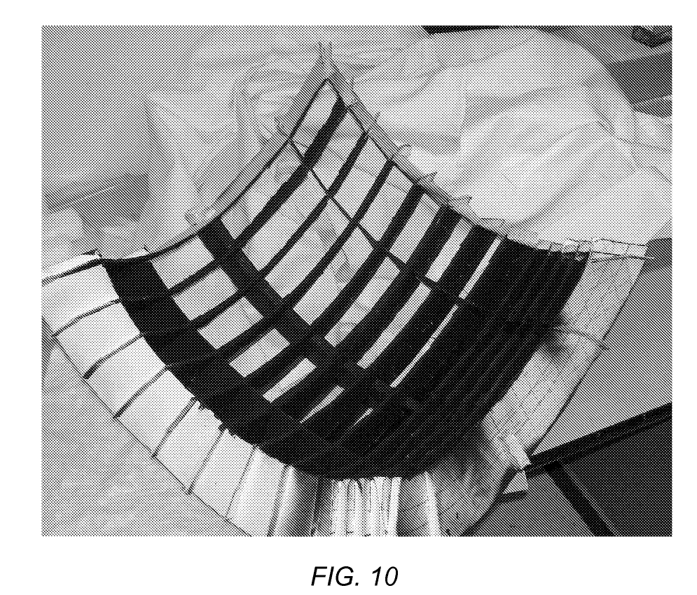

FIG. 10 illustrates fins of a rectangular shaped plaque 100 in accordance with one or more embodiments of the present technique.

Turning to FIG. 6C, an overview of an alternate embodiment of an ophthalmic plaque is shown. Rim 611 shown here is irregularly shaped (compare with rim 111 superimposed over rim 611). In an embodiment, the shape of rim 611 may be configured to maximize the number of radiation seeds that can be accommodated in the cavity defined by rim 611 and the base of the housing.

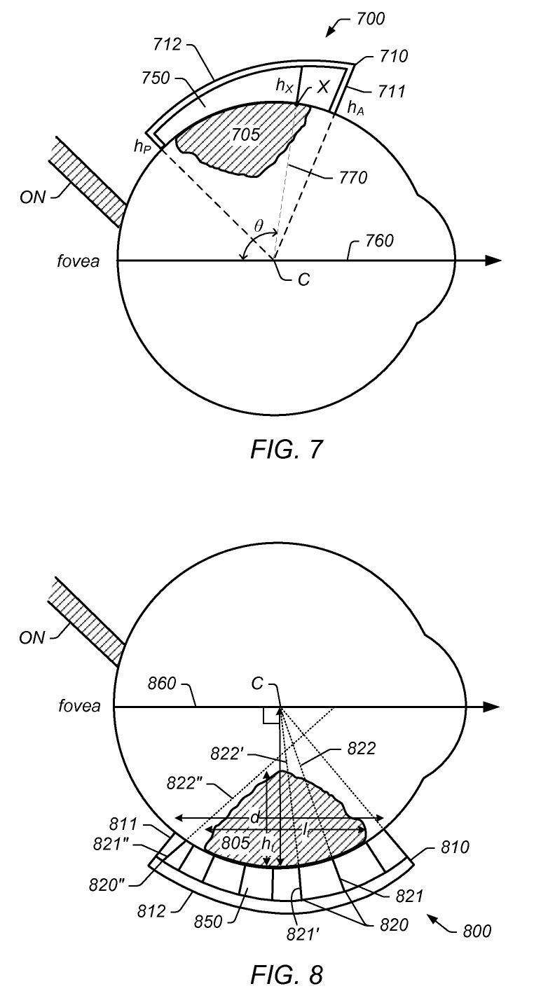

Turning now to FIG. 7, an alternate embodiment of the placement of ophthalmic plaque 700 in relation to choroidal melanoma tumor 705 is shown. Ophthalmic plaque 700 may include housing 710 comprising rim 711 and concave base 712 coupled to the perimeter of the rim, thereby defining cavity 750. In one embodiment, the height of rim 711 may vary at different points along the perimeter thereof. In the embodiment depicted in FIG. 7, the height of rim 711 at the posterior portion of housing 710 (defined as h_{P}) may be different from (i.e., less than or greater than) the height of rim 711 at the anterior portion of housing 710 (defined as h_{A}). The term “posterior” in the present context is relative to visual axis 760, and refers to the portion of ophthalmic plaque 700 that is oriented toward the posterior portion of the eye (i.e. facing the fovea/optic nerve) during use. In some embodiments, the term refers to at least about 75%, at least about 50%, or at least about 25% of the housing facing the posterior portion of the eye. Likewise, the term “anterior” in the present context refers to the portion of ophthalmic plaque 700 that is oriented toward the anterior portion of the eye (i.e. facing the fovea/optic nerve) during use. In some embodiments, the term refers to at least about 75%, at least about 50%, or at least about 25% of the housing facing the anterior portion of the eye.

In an embodiment, the height of rim 711 may become taller toward the anterior portion of the ophthalmic plaque. In one embodiment, the height of rim 711 at a point X on the surface of the eye (see FIG. 7), which is denoted hₓ, may generally be described by formula (I):

hₓ=k•sinθ+h_{P} (I);

where k is the increase in height per increase in sine value of θ to point X as θ moves from the posterior to the anterior portion of housing 710;

where t represents the angle between visual axis 760 and line 770 passing through center C of the eye and point X on the surface of the eye.

In an embodiment, the value of hₓ maybe in the range of about 1 mm to about 4.5 mm, or about 2 mm to about 3.5 mm.

FIG. 8 shows an alternate embodiment of the placement of ophthalmic plaque 800 in relation to choroidal melanoma tumor 805 is shown, where the basal diameter of the tumor is denoted by lₜ, and the height of the tumor is denoted by hₜ.

In an embodiment, ophthalmic plaque 800 may include housing 810 comprising rim 811 and concave base 812 coupled to the perimeter of the rim, thereby defining cavity 850. The diameter d of housing 810 is greater that the basal diameter lₜ of the tumor being treated. In an embodiment, cavity 850 may include a plurality of substantially parallel fins 820 positioned therein. FIG. 8 shows a cross sectional view of an eye, along the visual axis. In this embodiment, the plurality of substantially parallel fins 820 are oriented substantially perpendicular to visual axis 860. In an embodiment, the plurality of substantially parallel fins 820 may comprise a first set of a second set of fins, as described above. Though not shown in this particular view of the embodiment, it will be readily appreciated by the skilled practitioner that ophthalmic plaque 800 may include a further plurality of fins oriented substantially parallel to visual axis 860.

In one embodiment, each fin 820 may be individually angled in cavity such that planar surface 821 is substantially parallel with line 822 extending from the center C of the eye to fin 820. Likewise, fin 820′ may be angled such that planar surface 821′ is substantially parallel with line 822′ extending from the center C of the eye to fin 820′. Without being bound by any one particular theory or mechanism of action, it is believed that by angling at least a portion of the fins such that the planar surfaces thereof are aimed toward the center of the eye, the amount of irradiative energy that is delivered to substantially adjacent healthy tissue may be minimized, while still administering sufficient radiation to the tumor.

In one embodiment, substantially all of the fins comprising a set of fins oriented perpendicular to the visual axis may be angled as described above. In another embodiment, only a portion of the fins may be angled thus, while at least a potion of the remaining fins are angled differently. For example, in an embodiment, at least one fin 820″ located toward the posterior end of the of housing 810 may be angled such that planar surface 821″ is substantially parallel to line 822″ that intersects visual axis 860 anterior to center C of the eye. Angling at least one fin 820″ thus may further reduce the amount of radiation delivered to highly sensitive macula (containing the fovea) and optic nerve at the posterior end of the eye chamber, while still ensuring adequate irradiation of the entire tumor. The angled that fin 822″ may be positioned will vary depending on the size and the location of the tumor in relation to these tissues, and may be determined clinically using a variety of procedures typically used to map choroidal melanomas.

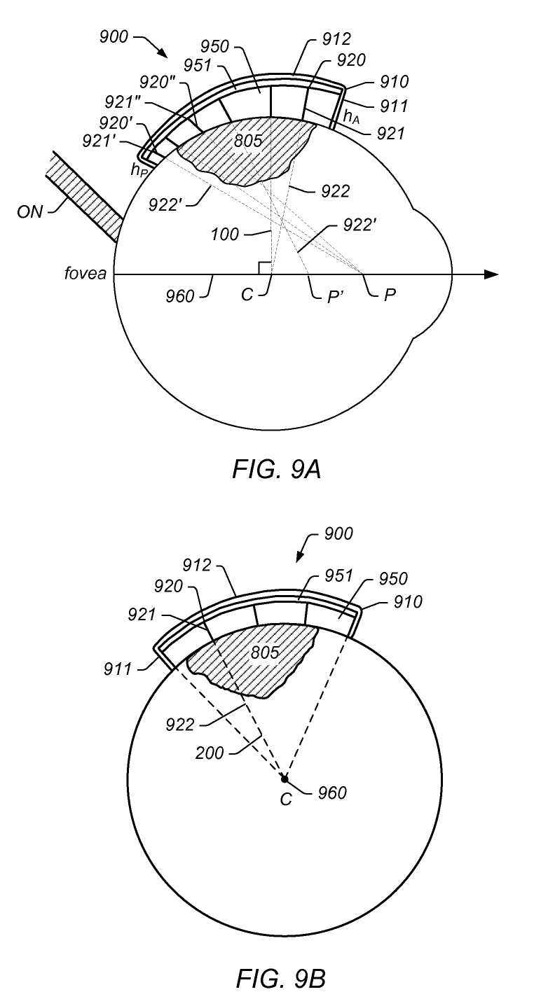

FIG. 9A shows an embodiment that include one or more of the features described above with regard to FIGS. 7 and 8. In the embodiment depicted in FIG. 9A, ophthalmic plaque 900 may include housing 910 comprising rim 911 and concave base 912 coupled to the perimeter of the rim, thereby defining cavity 950. The height h_{A} of rim 911 at the anterior portion thereof may be greater than the height h_{P} at its posterior portion. Cavity 950 may include a plurality of substantially parallel fins 920 positioned therein. In an embodiment, ophthalmic plaque 900 may further include seed carrier 951 positioned between base 912 and fins 920. FIG. 9A shows a cross sectional view of an eye, along the visual axis. In this embodiment, the plurality of substantially parallel fins 920 are oriented substantially perpendicular to visual axis 960. In an embodiment, the plurality of substantially parallel fins 920 may comprise a first set or a second set of fins, as described above.

In an embodiment, one or more fins 920 may be angled such that planar surface 921 is substantially parallel with line 922 that passes from fin 920 center to point C located at about the center of the eye. In one embodiment, the majority of fins comprising a set of fins may be angled thus. In one embodiment, only a portion of the fins comprising a set of fins may be angled thus. In one embodiment, at least a portion of the fins positioned toward the anterior portion of housing 910 may be angled thus.

In one embodiment, one or more fins 920′ may be angled such that planar surface 921 is substantially parallel with line 922′ that extents from fin 920′ and intersects visual axis 960 at point P, located anterior to point C at about the center of the eye. In one embodiment, the majority of fins comprising a set of fins may be angled thus. In one embodiment, only a portion of the fins comprising a set of fins may be angled thus. In one embodiment, at least a portion of the fins positioned toward the posterior portion of housing 910 may be angled thus.

FIG. 9B is an alternate cross-sectional view of the embodiment depicted in FIG. 9A. In this view, visual axis 960 is perpendicular to the page of the page. In the embodiment depicted in FIG. 9B, ophthalmic plaque 900 may include housing 910 comprising rim 911 and concave base 912 coupled to the perimeter of the rim, thereby defining cavity 950. Cavity 950 may include a plurality of substantially parallel fins 920 positioned therein. In an embodiment, ophthalmic plaque 900 may further include seed carrier 951 positioned between base 912 and fins 920. FIG. 9B shows a cross sectional view of an eye, perpendicular to the visual axis. In this embodiment, the plurality of substantially parallel fins 920 are oriented substantially parallel to visual axis 960. In an embodiment, the plurality of substantially parallel fins 920 may comprise a first set or a second set of fins, as described above. In an embodiment, the majority of fins comprising the set of fins oriented substantially parallel to the visual axis may be angled such that planar surface 921 is substantially parallel to line 922 extending from fin 920 an passing through point C located approximately at the center of the eye.

The device described herein may be used in the treatment of an eye or another area of the body. In one embodiment, a method of using one or more embodiments of an ophthalmic plaque descried herein may include: assessing the application, selecting an appropriate plaque, preparing the plaque, affixing the plaque to the affected region, leaving the plaque affixed to the affected region for a sufficient period of time, and removing and/or disposing of the plaque.

In one embodiment, assessing the application may include assessing or otherwise determining the current status of the tumor or aliment to be treated. For example, in one embodiment, a practitioner (e.g., a doctor) may assess the size and extent of the tumor on the eye to determine whether treatment with a plaque is suitable. Further, a practitioner may perform a biopsy or similar technique to determine the type of tumor. Selection of the plaque may be determined at least based on the assessment.

In one embodiment, selecting the appropriate plaque may include determining a size of the plaque suitable to treat the affected region. For example, in one embodiment selection of the appropriate plaque may include a practitioner selecting a plaque having a diameter sufficient to completely cover the affected region and/or provide a sufficient band of coverage surrounding the affected region. Further, an embodiment may include the selection of the radiation seed. For example, in one embodiment, a practitioner may select a larger dosage of radiation seed to treat a relatively large sized tumor and a smaller dosage of radiation seed to treat a relatively small sized tumor. In yet another embodiment, selecting the appropriate plaque may include selecting an appropriate type of radiation source. For example, a practitioner may select one or more of ¹²⁵I, ⁶⁰Co, ²²²Rn, ¹⁰⁶Ru, ¹⁹²Ir and ¹⁰³Pd.

In one embodiment, preparing the plaque may include assembling or otherwise preparing the plaque for use. In certain embodiments, assembling the plaque may include affixing the radiation seed to the upper surface of the base. In one embodiment, the radiation seed is affixed to a seed carrier and the seed carrier and/or the radiation seed is affixed to the upper surface of the base. In other embodiment, the plaque may be pre-prepared. For example, in one embodiment, one or more prepackaged plaques may be available for use. In such an embodiment, the practitioner may simply select the prepackaged plaque and remove it from its package for use. For example, in one embodiment, the radiation seed may be preassembled to the plaque at a radiopharmacy and delivered to the medical facility for use by the practitioner.

In one embodiment, affixing the plaque to the affected region includes placing the plaque at or near the affected region to substantially cover the affected region. For example, the plaque is affixed or otherwise held in place on a surface of the eye to completely cover the tumor. In certain embodiments, the plaque is positioned such that the fins are oriented to substantially focus onto the tumor, the radiation emitted by the radiation seeds. In one embodiment, affixing the plaque includes suturing the plaque to the surface of the eye. The sutures may be provided through eyelets of the plaque.

In one embodiment, leaving the plaque affixed to the affected region for a sufficient period of time includes allowing the plaque to remain affixed to the eye for period of time sufficient to provide a suitable dosage of radiation to the affected region. For example, the plaque may remain affixed to the eye for several minutes, hours, days, weeks, months, or more.

In one embodiment, removing and/or disposing of the plaque includes separating the plaque from the eye, and disposing of the plaque in accordance with regulations related to disposal of radioactive materials. For example, in one embodiment, the suture or other affixing device is serrated to release the plaque from the surface of the eye, and the plaque and the radiation seed(s) are disposed of in accordance with regulations. In one embodiment, the plaque may be disassembled such that the plaque and radiation seed(s) may be separately disposed of. In certain embodiments, the plaque may be reconditioned for future reuse.

Embodiments set forth herein may also be useful for the treatment of retinoblastoma and other intraoccular tumors.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

1. A device suitable for treating an eye, comprising:
a housing, the housing comprising
a base; and
a rim coupled to the perimeter of the base, wherein the base and the rim at least partially define a cavity in the housing, wherein the cavity is configured to accept one or more radiation seeds; and
a plurality of fins at least partially residing within or proximate the cavity of the housing, wherein at least a portion of the fins are configured such that radiation emitted from one or more radiation seeds positioned in the cavity is substantially directed toward a center portion of the eye during use.

2. The device in accordance with claim 1, wherein the fins comprise a first set in which at least two fins are substantially parallel, and a second set in which at least two fins are substantially parallel.

3. The device in accordance with claim 2, wherein the first set of fins and the second set of fins are oriented substantially perpendicular to each other.

4-10. (canceled)

11. The device in accordance with claim 1, wherein the base comprises a concave surface.

12. (canceled)

13. The device in accordance with claim 1, at least a portion of the housing comprises a shielding metal.

14-24. (canceled)

25. The device in accordance with claim 1, wherein the fins comprise at least one metal.

26. The device in accordance with claim 1, wherein the fins are plated with a shielding metal.

27-31. (canceled)

32. The device in accordance with claim 1, wherein an edge of at least a portion of the fins is curved, wherein the curve is substantially complementary to a surface of an eye.

33. The device in accordance with claim 1, wherein at least a portion of the fins are angled such that, during use, the axis thereof is substantially parallel to a radius of the eye.

34. The device in accordance with claim 1, wherein the radiation seed is configured to be oriented substantially perpendicular to a visual axis of the eye during use.

35. The device in accordance with claim 1, further comprising a silastic seed carrier positioned between the base of the housing and the fins.

36. The device in accordance with claim 1, further comprising a plurality of suture attachment means coupled to the housing.

37. The device in accordance with claim 1, wherein at least a portion of the fins are configured such that radiation emitted from one or more radiation seeds positioned in the cavity is substantially directed toward a center of the eye during use.

38. A method of treating an eye, comprising:
providing radiation to the eye via an eye treatment device, wherein the eye treatment device comprises:
a housing, the housing comprising
a base; and
a rim coupled to the perimeter of the base, wherein the base and the rim at least partially define a cavity in the housing, wherein the cavity is configured to accept one or more radiation seeds; and
a plurality of fins at least partially residing within or proximate the cavity of the housing, wherein at least a portion of the fins are configured such that radiation emitted from one or more radiation seeds positioned in the cavity is substantially directed toward a center portion of the eye during use.

39. A method, comprising:
affixing a treatment device to a surface of an eye, wherein the treatment device comprises a plurality of fins disposed between one or more radiation seeds housed within the treatment device and the surface of the eye, and wherein at least two of the fins are configured such that radiation emitted from one or more radiation seeds positioned in the cavity is substantially directed toward the center portion of the eye during use.

40-45. (canceled)
